(19) United States
(12) Patent Application Publication
(10) Pub. No.: US 2026/0048001 A1
(43) Pub. Date: Feb. 19, 2026

(54) SKINCARE COMPOSITION CONTAINING CASHEW APPLE EXTRACT

(71) Applicant: R & D Global Consultants Pvt Ltd, Thane (IN)

(72) Inventors: Deepak Kisandas Rohra, Mumbai (IN); Renuka Thergaonkar, Thane (IN)

(21) Appl. No.: 19/269,596

(22) Filed: Jul. 15, 2025

(30) Foreign Application Priority Data
Jul. 15, 2024 (IN) 202421053667

(51) Int. Cl. A61K 8/9789; A61K 8/34; A61K 8/37; A61K 8/44; A61K 8/60; A61K 8/81; A61Q 19/00; A61Q 19/02; A61Q 19/08

(52) U.S. Cl. CPC A61K 8/9789; A61K 8/34; A61K 8/342; A61K 8/345; A61K 8/37; A61K 8/44; A61K 8/60; A61K 8/8152; A61Q 19/007; A61Q 19/02; A61Q 19/08

(57) ABSTRACT

The present invention provides topical skincare compositions containing extract of cashew apple. Particularly, the skincare composition of the invention is helpful in imparting multiple skin-enhancing benefits including anti-aging and collagen-boosting effects, antioxidant activity, skin whitening or brightening, deep moisturizing activity, and sculpting or anti-cellulite action. The composition is also formulated to be gentle on the skin, exhibiting low irritation potential, thereby making it suitable for daily and prolonged use.

### FIELD OF THE INVENTION

The present invention relates to skincare compositions containing extract of cashew apple. Particularly, the skincare composition of the invention pertains to topical cosmetic or dermatological compositions that exhibit multiple skin-enhancing benefits including anti-aging and collagen-boosting effects, antioxidant activity, skin whitening or brightening, deep moisturizing activity, and sculpting or anti-cellulite action. The composition is also formulated to be gentle on the skin, exhibiting low irritation potential, thereby making it suitable for daily and prolonged use.

### BACKGROUND OF THE INVENTION

Cashew (Anacardium occidentale L.) belongs to the family Anacardiacea. It was originally introduced into India by the Portuguese during the 16^{th} century. Runjala et al (Cashew apple (Anacardium occidentale L.) therapeutic benefits, processing and product development: An overview; Suneetha Runjala and Dr. Lakshman Kella; The Pharma Innovation Journal 2017; 6(7): 260-264) discloses that the fruit of the cashew tree is an accessory fruit (sometimes called a pseudo carp or false fruit). What appears to be the fruit is an oval or pear-shaped structure, a hypocarpium that develops from the pedicel and the receptacle of the cashew flower. Called the cashew apple, better known in Central America as maranon, it ripens into a yellow and/or red structure about 5-11 cm long. It is edible, and has a strong “sweet” smell and a sweet taste. Cashew apples possess anti-bacterial properties and have been proven to be effective in treating stomach ulcers and gastritis. Its juice is rich in vitamin C and has an anti-scurvy effect. Cashew apple juice is widely utilized in the cosmetic industry due to the presence of antioxidants and is used in the preparation of various creams and shampoos. Cashew extract contains anarcardic acid which is an antioxidant and has been shown to limit the pigmentation effects of aging and to eradicate the cancer cells. Fruit of the cashew tree is used to treat infant's thrush and sore mouth.

Cashew is essentially a tropical crop, grows best in the warm, moist and typically tropical climate. The distribution of cashew is restricted to altitudes below 700 m where the temperature does not fall below 200° C. for prolonged periods, although it may be found growing at elevation up to 1200 m. It is best adopted to the coastal regions. The cashew is hardy and drought resistant, but it is damaged by frost.

Runjala et al also states that many properties of fresh cashew apple juice have been proposed and continue to be proposed for its therapeutic values like anti-oxidant, anti-fungal, antibacterial, anti-tumor, anti-inflammatory, anti-mutagenic activities. Anacardic acids, along with (E)-2-hexenal have been found to exhibit broad antimicrobial activity. The total anacardic acid content in cashew apple is 1.1 g/kg and is suggested for its utilization in functional food formulations. Anacardic acids are used in cosmetics for disinfection. Ascorbic acid and tannic acid contribute considerably to the overall antioxidant capacity of the cashew apple juice. The antioxidant activity of cashew apple is correlated well to the quantities of alkyl phenols, anacardic acids and cardols. The antioxidant activity of anacardic acid-1 (6-[8(Z), 11(Z), 14-pentadecatrienyl] salicylic acid) relates to the inhibition of superoxide generation (IC50=0.04 mM) and xanthine oxidase activity (IC50=0.30 mM). The presence of chemically active components (ascorbic acid, anacardic acid, carotenoids, condensed tannins, quercetin, and other phenolic compounds) in fresh and processed cashew apple juice are responsible for the anti-mutagenic mechanism, which is shown to be involved in stimulation of DNA repair or reversion of DNA damage. Due to lack of awareness about the nutritional values of cashew apples and suitable technology for preservation, thousands of tons of cashew apples are wasted every year. Runjala et al provides the chemical composition and nutritive value of cashew apples.

Cashew apple is rich in vitamin C, sugars and contains considerable amount of tannins and minerals mainly calcium, iron and phosphorus. Ripe cashew apple contains 88 to 90% moisture, 12.3% carbohydrates, 0.2% proteins, 0.1% fat, 0.9% crude fibre, 10.0 mg/100 g Calcium, 0.2 mg/100 g iron, 261 mg/100 g Vitamin C, 0.2% minerals, 0.02 mg/100 g thiamine, 0.5 mg/100 g riboflavin, 0.4 mg/100 g nicotinic acid, 39.0 IU vitamin A, 10.37% total sugars, 9.61% reducing sugars, 0.32% titratable acidity, 199.08 mg/100 g ascorbic acid, and 0.15% tannins.

Despite its richness in bioactives, commercial utilization of cashew apple remains limited due to lack of preservation technologies and market awareness, leading to substantial post-harvest waste each year. While some cosmetic formulations have used cashew derivatives, these often relate to seed or shell extracts, and not to the application of extracts obtained from cashew apple, directly on the skin.

U.S. Pat. No. 8,747,927B2 claims the use of Myrciaria cauliflora (jaboticaba) fruit pulp wherein the composition is described to inhibit COX-1 and TNF-C activity in skin cells, while also increasing hyaluronic acid synthesis in said cells. The specification contemplates the use of combination of jaboticaba fruit pulp and cashew fruit pulp to provide the claimed skin effects, but not enough data is available to understand the impact or effect of such application on various parameters like cellulite reduction to provide sculpting, anti-aging effect, effect on moisture levels of the skin, and/or skin brightening. While Example 1 is worded to show synergy of the combination of jaboticaba fruit pulp and cashew apple fruit pulp, the actual effect on skin is not shown. Most other examples merely provide the test methods but do not provide results. This document, therefore, does not clearly teach the effects and/or benefits on actual use.

U.S. Pat. No. 10,028,970B2 discloses use of hydroalcoholic extracts of cashew apple fruit in treating various conditions including reduction of fat storage. It relates to dietary supplement, nutraceutical or food compositions containing cashew apple extract that are meant for oral consumption. Nothing therein relates to topical compositions of cashew apple extract that can provide benefits to the skin.

US20200069754A1 (similar disclosures in U.S. Pat. Nos. 11,497,784B2 and 10,967,022B2) focusses on melanin control and anti-ageing effects on the skin, using the antioxidant activity of cashew seed coat extract, or cashew testa extract. It must be noted that the contents of cashew testa extract (see Sruthi et al, Food Chemistry Advances, Volume 3, December 2023, 100390) are significantly different from those of the cashew apple extract. Further, nothing in this document teaches, suggests or motivates the use of cashew apple extract for formulating skincare compositions for topical application, that can help in skin sculpting, cellulite reduction, skin brightening, skin moisturisation and wrinkle control. It would not be possible to even assume, therefore, that the cashew apple extract would have similar effects as that of the cashew testa extract.

US20210346454A1 describes a method of inhibiting oxidation-induced DNA damage by using seed coat extract of cashew apple, i.e. cashew testa, as a composition to be administered orally. However, it does not mention topical usage of cashew apple fruit extract on the skin, nor does it disclose use in any skin benefits as taught herein.

JP2009155259A focusses on inhibitory action of the cashew apple on various enzymes resulting in various applications of cashew apple including, elastase inhibitory action that suppresses aging and wrinkles. However, the document does not disclose topical use of cashew apple extract specifically for reducing cellulite, neither does it demonstrate any improvement in skin elasticity. In the absence of any such teaching it would not be possible to envisage the skin benefits taught herein through topical application of the cashew apple extract.

CN112638179A discloses use of seed coat extract of cashew nuts for skin lightening. However, it does not mention usage of cashew apple extract for providing skin benefits.

Ganbold, M., Takahashi, S., Kakui, O. et al. Inhibitory effects of cashew Anacardium occidentale L. kernel, apple, and shell extracts on lipid accumulation and adipogenesis in 3T3-L1 adipocytes. Sci Rep 15, 1644 (2025). https://doi.org/10.1038/s41598-025-85727-3 discloses that ethanolic extract of dried cashew apple was found to inhibit lipid accumulation in 3T3-L1 adipocytes. However, it does not explicitly mention topical applications of cashew apple extracts and its impact on cellulite reduction and/or enhanced skin elasticity.

Thus, while a lot is known about the components of the cashew apple fruit, and about use of the same in various ailments and conditions, there is no reference disclosing safe and acceptable topical formulations containing cashew apple extract, which provide multifunctional benefits to the skin.

An online advertisement in the Happi magazine, discloses the inventor's formulation with cashew apple extract, which provides a sculpting and collagen boosting effect. The formulation is described to be rich in natural ascorbic acid, tannins and flavonoids contained in the cashew apple extract. The present application bears priority from Indian priority application number IN 202421053667 filed prior to the online appearance of this advertisement.

### SUMMARY OF THE INVENTION

The present invention provides a composition for topical application comprising:
(i) cashew apple extract in an amount ranging from about 0.5% to about 50% by weight of the composition; and
(ii) one or more suitable excipients selected from the group consisting of solvents, polymers, oils, penetration enhancers, humectants, thickening agents, preservatives, antioxidants, chelating agents, fragrances, colours, emollients and pH adjusting agents.

In one embodiment, the topical composition of the present invention can be safely applied at least once daily for at least 15 days.

The topical compositions of the present invention may be in the form of cream, gel, lotion, solution, tincture, ointment, mousse, serum, spray, mist oil, powder or paste, suitable for regular application to the skin.

In one embodiment, the topical composition is non-reactive to the skin and imparts anti-cellulite activity for skin sculpting.

In another embodiment, the topical composition provides anti-tyrosinase activity for skin brightening.

In yet another embodiment, the composition provides collagen stimulation to the skin thereby providing anti-aging effect.

In another embodiment, the composition exhibits free radical scavenging activity that provides antioxidant effect to the skin.

In still another embodiment, the composition provides increased skin moisturization when applied daily for 15 days.

In a highly preferred embodiment, the topical composition of the present invention provides a method for enhancing overall skin appearance, wherein topical application to the skin at least once daily for a period of at least 15 days results in reduction in the appearance of cellulite, improvement in skin brightness, enhanced skin moisturization, stimulation of collagen production and reduced aging of the skin.

### BRIEF DESCRIPTION OF THE FIGURES

FIG. 1 provides cytotoxicity evaluation chart of the cashew apple extract of the present invention at different concentrations.

FIG. 2 provides Adipocyte Lipolytic Assay with differentiated adipocytes using 50% concentration of Cashew apple extract of the present invention.

FIG. 3A provides pictorial representation of Sebutape test of Serum A and Serum B of Example 1.

FIG. 3B provides microscopic evaluation of strip from female subject after 20 minutes where (a) provides observation for Blank, and (b) provides that for Test Serum A.

FIG. 3C provides microscopic evaluation of strip from male subject after 20 minutes where (a) provides observation for Blank, and (b) provides that for Test Serum B.

FIG. 3D provides microscopic evaluation of strip from female subject after 3 hours where (a) provides observation for Blank, and (b) provides that for Test Serum A.

FIG. 3E provides microscopic evaluation of strip from male subject after 3 hours where (a) provides observation for Blank, and (b) provides that for Test Serum B.

### DETAILED DESCRIPTION OF THE INVENTION

The term formulation or composition may be used interchangeably throughout the specification.

The terms “cashew apple fruit”, “cashew fruit” and “extract” may be used interchangeably, and they all relate to the cashew apple extract of the present invention, which is extracted and used in the manner described herein.

The present invention relates to a skincare composition comprising cashew apple extract in an amount ranging from about 0.5% by weight to about 50% by weight of the composition, along with one or more suitable excipients. The composition is designed for topical application to the skin at least once daily, preferably for a duration of 15 days or more. This results in visible cosmetic benefits, most notably the reduction of cellulite and enhancement of skin firmness and elasticity. Additionally, the composition provided skin moisturising, anti-oxidant and skin regeneration effects. These functional properties are a result of definitive results in skin studies conducted with the cashew apple extract.

In various embodiments, the cashew apple extract may be present in varying concentrations within the total composition. In one embodiment of the present invention, the cashew apple extract is present in an amount ranging from about 0.5% w/w to about 1% w/w of the total composition. In another embodiment of the present invention, the cashew apple extract is present in an amount ranging from about 1% w/w to about 10% w/w of the total composition. Yet in another embodiment of the present invention, the cashew apple extract is present in an amount ranging from about 10% w/w to about 20% w/w of the total composition. Yet in another embodiment of the present invention, the cashew apple extract is present in an amount ranging from about 20% w/w to about 30% w/w of the total composition. Yet in another embodiment of the present invention, the cashew apple extract is present in an amount ranging from about 30% w/w to about 40% w/w of the total composition. Yet in another embodiment of the present invention, the cashew apple extract is present in an amount ranging from about 40% w/w to about 50% w/w of the total composition.

The cashew apple extract of the present disclosure contains phytochemicals such as flavonoids, tannins, proteins and triterpenoids. Flavonoids are particularly useful for maintaining healthy circulation. In addition to being potent antioxidants, some flavonoids have anti-allergic, anti-oxidant and anti-inflammatory activity. Tannins are polyphenolic compounds that are astringents, making them useful for tightening up loose tissue, such as that which is found in varicose veins. Proteins help in the functioning of cells. Triterpenoid compounds have proven anti-inflammatory, antimicrobial and anti-tumor activity on the skin. The anti-inflammatory activity protects the connective tissue of the skin by reducing the expression of pro-inflammatory cytokines and suppressing enzymes that degrade collagen and other structural support proteins of the skin. Its purported effects are strengthening of tissues surrounding fat-storage cells, to stimulate collagen in the weakened walls of sub-dermal veins.

The cashew apple extract of the present invention may be incorporated in various skincare and cosmetic compositions such as creams, gels, lotions, solutions, tinctures, ointments, mousse, serums, sprays, mists, oils, powders, pastes and the like.

The cashew apple extract used in the compositions of the present invention may be obtained by processes using various solvents including water, ethanol, propanol, isopropyl alcohol, butanol, acetone, petroleum ether, ethyl acetate, pentane and mixtures of these solvents such as hydroalcoholic solutions. Common methods used for obtaining the cashew apple extract using these solvent(s) include soxhlet extraction, ultrasonic-assisted extraction, microwave-assisted extraction, Pressurized Liquid Extraction, Supercritical Fluid Extraction, and Enzymatic Treatment. These techniques leverage the solubility differences between desired compounds and the fruit matrix to isolate valuable components. Several other methods can also be used for the extraction including pressing (hydraulic, screw, or basket press) and blending. Temperature is critical during the extraction process, and care needs to be taken to ensure that the extract and the fruit does not undergo fermentation.

In an embodiment of the present invention, the composition comprises suitable excipients. These include one or more of solvents, polymers, oils, penetration enhancers, humectants, thickening agents, preservatives, antioxidants, chelating agents, fragrances, colors, emollients, pH adjusting agents, and the like.

In one embodiment, solvents are selected from the group comprising water, isopropyl alcohol, ethanol, benzyl alcohol, silicones, liquid paraffin, fatty acid esters, isopropyl palmitate, isopropyl myristate, propenediol, glycerine, caprylic/capric triglyceride, medium chain triglycerides, vegetable oil, diethyl succinate, diheptyl succinate, dihexyl succinate, diisooctyl succinate, neopentyl glycol diheptanoate, C₁₃-C₁₆ isoalkane, propylene glycol dibenzoate, isododecane, and a combination thereof. Solvents may be present in an amount ranging from about 60% v/w to about 85% v/w of the total composition. These solvents aid in solubilizing the active ingredients and maintaining formulation consistency.

The thickening agents are used in the composition for obtaining the desired texture of the composition with the desirable rheology. In an embodiment of the present invention, the thickening agents may be selected from the group comprising cetyl alcohol, stearyl alcohol, cetearyl alcohol, myristyl alcohol, beeswax, carnauba wax, candelilla wax, xanthan gum, guar gum, carrageenan, gum arabic, locust bean gum, pectin, agar, hydroxyethylcellulose, methylcellulose, carboxymethyl cellulose (CMC), hydroxypropyl methylcellulose (HPMC), carbomer, acrylic crosspolymers, sodium polyacrylate, corn starch, potato starch, aluminium starch, octenylsuccinate, sodium polyacrylate starch, silica, bentonite, magnesium aluminum silicate, gelatin, aloe vera and mixtures thereof. These agents may be present in an amount ranging from about 0.1% w/w to about 5% w/w of the total composition.

Preservatives are added to the product for maintaining the shelf life of the product during storage and provide antimicrobial efficacy required to prevent microbial growth in the product. Suitable preservatives that may be used in the composition may be selected from the group comprising methyl paraben, ethyl paraben, propyl paraben, butyl paraben, benzoic acid, sodium benzoate, sorbic acid, potassium sorbate, diazolidinyl urea, imidazolidinyl urea, hydantoin, phenoxyethanol, benzyl alcohol, glyceryl ester, methylheptyl glycerine, caprylyl glycol, potassium hydrogen caprylhydroxamate, glyceryl undecylenate, glyceryl heptanoate, capryl hydroxamic acid, phenylethyl alcohol, ethylhexyl glycerine and mixtures thereof. The preservative may be present in the composition in an amount ranging from about 0.05% w/w to about 1% w/w of the total composition.

To enhance dermal penetration of the cashew apple extract, the composition incorporates penetration enhancers. The penetration enhancers may be selected from the group comprising caprylic/capric monoglyceride, Piper nigrum (black pepper) oil extract, sophorolipid, isopropyl myristate, 2-pyrrolidone, propylene glycol, oleic acid, myristic acid, urea, isopropyl palmitate, ethanol, propenediol, sodium lauryl sulfate, benzalkonium chloride, maltodextrin, cyclodextrin, menthol, limonene, neem oil, eucalyptus oil, basil oil, nutmeg oil, sunflower oil, glycerine, urea, cetyl alcohol, cetostearyl alcohol, oleyl alcohol, betaine, mangiferrin and mixtures thereof. These agents are present in an amount ranging from about 0.1% w/w to about 5% w/w, preferably from about 0.5% w/w to about 2.5% w/w of the total composition.

Maintaining skin pH and balance is pertinent to any cosmetic formulation. The pH adjusting agent is added in an amount such that it helps in obtaining a pH between pH 5 to 9, so as to be compatible with the skin pH. The pH adjusting agents that may be used in the compositions of the present invention may be selected from the group comprising citric acid, lactic acid, sodium hydroxide, potassium hydroxide, triethanolamine, sodium bicarbonate, arginine, aminomethyl propanol and mixtures thereof. In one embodiment of the present invention, the pH adjusting agent is triethanolamine.

Chelating agents help stabilize the formulation by binding metal ions that may otherwise catalyze degradation, and may be selected from the group comprising disodium EDTA, sodium gluconate and mixtures thereof. In one embodiment of the present invention, the chelating agent is disodium EDTA in an amount ranging from about 0.05% w/w to about 0.15% w/w of the total composition.

Emollients help in creating stable compositions and softening the skin. The emollients suitable for use in the compositions of the present invention may be selected from the group comprising shea butter, cocoa butter, lanolin, mineral oil, petrolatum, squalene, jojoba oil, castor oil, safflower oil, rosehip seed oil, avocado oil, caprylic triglyceride, capric triglyceride, isopropyl palmitate, isopropyl myristate, stearic acid, glyceryl stearate, octyl stearate, dimethicone, cyclomethicone, propylene glycol, stearic acid, palmitic acid, myristic acid, lauric acid and mixtures thereof. Emollients may be present in an amount ranging from about 1% w/w to about 90% w/w of the total composition.

The compositions of the present invention may also include humectants to provide suitable hydration and moisturisation to the skin. These may be included in amounts ranging from about 1% w/w to about 25% w/w of the total composition, and may be selected from the group comprising trehalose, glycerine, sorbitol, propylene glycol, hyaluronic acid, urea, honey, panthenol, lactic acid, glycolic acid, sodium gluconate, sodium lactate, butylene glycol, hexylene glycol and mixtures thereof.

In a preferred embodiment of the present disclosure, the composition comprises about 20% w/w of cashew fruit extract as the primary active, along with about 2% w/w of a humectant, about 0.8% w/w of a thickening agent, about 0.25% w/w of a preservative, about 0.1% w/w of a chelating agent, and suitable amount of a solvent.

In a preferred embodiment, the humectant is trehalose, the thickening agent is polyacrylate crosspolymer-6, the preservative is methyl paraben, the chelating agent is disodium EDTA and the solvent is water. Additionally, the composition comprises about 2.24% w/w of a penetration enhancer containing Piper nigrum (black pepper) extract. This enhances transdermal delivery of the composition, thereby enhancing the efficacy of the composition. The black pepper extract is mainly formulated as an oil, obtained by general conventional methods available in the art. It is typically obtained through steam distillation of dried black peppercorns. This process involves introducing steam to the peppercorns, which vaporizes the volatile compounds, including the essential oil. The steam is then cooled, separating the oil from the water. In a highly preferred embodiment, the penetration enhancer comprises a blend of caprylic/capric monoglyceride, Piper nigrum oil extract, and sophorolipid, and is used in an amount ranging from about 2% v/w to about 10% v/w of the total composition. In one embodiment, the penetration enhancer is present in an amount of about 0.7% w/w of the total composition, and may be a mixture of enhancers.

In a preferred embodiment of the present invention, the composition is a mixture of an active phase containing the cashew apple extract, a blend of penetration enhancers, emollients and other acceptable excipients. The active phase comprises cashew apple extract, one or more solvents and one or more preservatives. The solvents are selected from the group comprising water, isopropyl alcohol or a combination thereof. The preservatives are selected from the group comprising sodium benzoate, potassium sorbate or a combination thereof. The active phase is present in an amount of about 2% w/w to about 10% w/w of the total composition. In a highly preferred embodiment, the active phase is present in an amount of about 5% w/w of the total composition. Additionally, the preferred embodiment comprises humectants selected from sodium gluconate, glycerine and a combination thereof, in an amount ranging from about 0.1% w/w to about 4% w/w of the composition. The composition also contains carbomer as a thickening agent in an amount ranging from about 0.1% w/w to about 1% w/w of the composition. The composition additionally contains water as a solvent to make up the remaining volume.

In a preferred embodiment, the composition comprises approximately 79.25% w/w water, 0.3% w/w carbomer, 0.2% w/w sodium gluconate, and 4% w/w glycerine as part of Phase A; Phase B consists of emollients selected from about 2.5% w/w stearic acid, 1.75% w/w cetostearyl alcohol, and 4% w/w isopropyl myristate; Phase C includes triethanolamine as a pH adjuster; while Phase D comprises 0.7% w/w of a penetration enhancer blend consisting of caprylic/capric monoglyceride, Piper nigrum oil extract and sophorolipid. The active phase E includes 5% w/w of a mixture containing cashew apple extract, water, glycerine, sodium benzoate, potassium sorbate, and isopropyl alcohol.

In one embodiment of the present invention, the composition of the present invention contains cashew apple extract as the sole active component, and does not include any other active agent or component. In another embodiment, the composition contains cashew apple extract in combination with one or more other natural extracts that provide synergistic skin improvement effects with cashew apple extract.

The compositions of the present invention may be in the form of various skincare and cosmetic compositions such as creams, gels, lotions, solutions, tinctures, ointments, mousse, serums, sprays, mists, oils, powders, pastes and the like. The composition provides multiple cosmetic effects, such as anti-aging, antioxidant, brightening, moisturizing, and sculpting of the skin.

The composition of the present invention imparts a protective effect against skin aging and oxidative stress by neutralizing reactive oxygen species (ROS) generated due to UV radiation, coupled with decreased antioxidant defence due to aging of the skin. At the cellular level, ROS causes mitochondrial dysfunction and disrupts the respiratory chain, leading to protein and lipid oxidation, collagen disorganization, and altering of the gene activity, resulting in upregulation of aging-related cytokines and matrix metalloproteinases (MMPs). The composition of the present invention helps prevent these deleterious effects by scavenging free radicals and improving the skin's antioxidant defence, thereby maintaining dermal integrity and delaying the onset of visible signs of aging such as fine lines and wrinkles.

The compositions of the present invention also exhibit anti-tyrosinase activity. Tyrosinase plays an important role in the formation of melanin pigments during melanogenesis. The enzyme tyrosinase catalyzes the oxidation of tyrosine (substrate) and certain diphenolic intermediate products to quinones, which polymerize to give rise to melanin. Melanin production is principally responsible for skin colour and plays an important role in the prevention of sun-induced skin injury. Under UV exposure, keratinocytes release signals to melanocytes, leading to increased melanin synthesis. The composition of the present invention inhibits tyrosinase activity, thereby reducing melanin production and transfer to keratinocytes, resulting in a brighter, even-toned skin appearance. At least once daily application of the present invention for at least 15 days was found to brighten the skin and reduced blemishes.

The composition of the present invention also improves skin hydration by reducing trans-epidermal water loss (TEWL), resulting in softer, smoother, and well-hydrated skin. TEWL is the process where water diffuses through the epidermis (the outer layer of skin) and evaporates from the skin's surface. Several factors can influence TEWL, including skin condition (e.g., dryness, eczema), environmental conditions (e.g., humidity, temperature), and even age. Essentially, the skin barrier function is impacted in such cases. The compositions of the present invention help in prevention or reduction of TEWL with the help of the cashew apple extract and the additives used. This is typically achieved due to the thin layer formed by the composition on the surface of the skin, wherein the various components included in the composition, and the cashew apple extract, together increase moisture levels in the skin by preventing surface loss of water from the skin. As a result, the skin remains hydrated, and the aging of the skin due to TEWL is controlled. At least once daily application of the present invention for at least 15 days was found to improve the hydration status of the skin.

Cellulite refers to the dimpled, lumpy appearance of skin caused by fat deposits pushing against connective tissue, commonly seen on the thighs, buttocks, and abdomen. It affects up to 90% of post-pubertal women and is influenced by factors such as age, genetics, skin thickness, and hormonal levels. Existing treatments like massage or surgical methods offer limited or temporary results and may not be suitable for all individuals, particularly those with underlying conditions. Cashew fruit extract of the present invention has shown tremendous activity in regeneration of the compromised skin structure that develops as a result of cellulite. The composition promoted lipolysis, i.e. the breakdown of fat stored in adipocytes. Reduction in adipose cell size aids in providing sculpting and anticellulite properties. By targeting local fat deposits and improving skin tone and firmness, the composition offers a non-invasive method for skin sculpting.

The compositions containing the cashew apple extract of the present invention may be packaged in suitable containers, made of materials that retain the efficacy and stability of the composition. The packaging used is such that it does not allow fermentation of the cashew apple extract, nor does it allow the composition to be exposed to environmental factors that might impact its physical and chemical integrity. The packaging may contain applicators that make it convenient to apply the composition to the affected area of the skin, such as spatulas. The packaging may be conventional in terms of bottles, jars, tubes that may be used, and can also be packed in bottles with metered dose dispensers, allowing defined quantity of the composition to be delivered. Accordingly, one or more metered dose amounts of the composition may be applied uniformly to the affected area, with uniform spreading and gentle rubbing.

The amount of composition containing the cashew apple extract of the present invention that is needed for achieving desirable results in cellulite reduction, skin brightening, anti-aging, in improving elasticity of the skin and/or in improving skin hydration, can be easily determined by skilled persons, such as dermatologists. The composition may be applied once or twice daily, depending on the need, for a period of at least 15 days to observe visible results.

The examples used herein are merely exemplary and should not be used to narrow the scope of the invention.

### EXAMPLES

### Example 1—Preparation of Cashew Apple Extract

The crushed cashew apple fruit (60 g) was placed in a covered container with water (40 ml), and allowed to stand over a period of 24 hours, while ensuring that it does not ferment, by maintaining temperature at 4-8° C. and continuous stirring. The resultant mixture was then strained and the liquid was clarified by filtration. The extract thus obtained was tested qualitatively for the chemical contents by the standard detection methods.

The results of analysis of the cashew apple extract are provided in Table 1 below.

TABLE 1

| | Total | Reducing | Titratable | | Ascorbic | | |
|---|---|---|---|---|---|---|---|
| | Sugars | Sugars | Acidity | | acid | Tannins | Proteins |
| Colour | % | % | % | pH | (mg/100 ml) | % | % |
| Golden | 3.2 | 0.14 | 0.15 | 5.22 | 78.06 | 0.108 | 0.235 |
| yellow | | | | | | | |

The extract was found to have a shelf life of approximately 3 years.

The extract was evaluated for its cytotoxicity on 3T3 cell line by Sulforhodamine B (SRB) assay. The efficacy of the extract was also evaluated on adipose cell line.

### Example 2—Preparation of Formulation I

The cashew apple extract of Example 1 was incorporated at a concentration of 20% in two serum base compositions. The extract was used without and with penetration enhancers (Serum A and Serum B (or formulation I), respectively). Commercially available Black Pepper extract was used as the penetration enhancer. The extract was then incorporated in Serum B. The compositions are disclosed in Table 2 below.

TABLE 2

| | | Serum B |
|---|---|---|
| | Serum A | (Formulation I) |
| Ingredients | (% w/w) | (% w/w) |
| Water | 76.85 | 74.61 |
| Cashew Fruit extract of | 20.00 | 20.00 |
| Example 1 | | |
| Trehalose | 2.00 | 02.00 |
| Polyacrylate Crosspolymer-6 | 0.80 | 0.80 |
| Methyl Paraben | 0.25 | 0.25 |
| Black pepper extract | — | 2.24 |
| Sodium EDTA | 0.10 | 0.10 |

The polyacrylate crosspolymer was first mixed with water and allowed to swell. Methyl paraben, Trehalose, Black pepper extract and sodium EDTA was then added to it. Finally, Cashew fruit extract of Example 1 was added to the mixture and it was stirred till a desired serum consistency was achieved. The compositions (or serums) were then tested for stability and tested on human volunteers for efficacy. The compositions were used on regular basis by 5 volunteers under observation and were found to have no adverse events during the study period. The evaluation of the product for safety was done by a dermatologist. The compositions were subjected to stability testing, (i) under accelerated stability conditions for 6 months, and (ii) at room temperature for a period of 1 year, and were found to be stable. No fermentation or other reaction was observed. No physical or chemical degradation was observed.

### Example 3: Sulforhodamine B (SRB) Assay

The study was conducted using several concentrations of cashew extract of Example 1, to check for the cell viability. The study was conducted asper the guidance provided in Skehan (P. Skehan et al: A New Colorimetric Cytotoxicity Assay for Anticancer-Drug Screening, Journal National Cancer Institute 82:1107-1112, 1990). The cashew apple extract of Example 1 was tested at concentrations of 0.25%, 0.5%, 0.75% and 1%. The stock solution was freshly prepared in sterile phosphate buffered saline (PBS). Further dilutions were prepared in sterile PBS. Higher concentrations were limited by solubility. Stock solution of 100 mg/ml of reference standard was prepared in sterile PBS. All concentrations were prepared by serial dilutions and were freshly prepared just before use in PBS.

Proliferation assessment of samples of cashew apple extract of Example 1 on Murine fibroblast cell line 3T3 by Sulforhodamine B (SRB) assay was designed to study the cytotoxicity potential of the cashew apple extract. The assay was performed in a 96-well tissue culture plate. The duration of the assay was 3 days. The cells were seeded into the plate, and after they were sub-confluent, they were treated with the test material (cashew apple extract of Example 1) for 24 hours. The cells were then fixed and stained with Sulforhodamine B, which is a protein stain. The color was read at 570 nm using a plate reader. The absorbance of near red medium was read, from which cell viability was calculated. The intensity of the color was directly proportional to the total protein, which is an indication of cell viability. The cytotoxicity/proliferation was scored on the basis of comparison with the untreated control. Reference standards were used to give an indication of cytotoxicity and/or proliferation.

The results are provided in the Table 3 below.

TABLE 3

| | Mean viabilities at different concentrations | | | |
|---|---|---|---|---|
| | Concentration | | | |
| | 0.25% | 0.5% | 0.75% | 1% |
| | (2.5 mg/ml) | (5 mg/ml) | (7.5 mg/ml) | 10 mg/ml |
| Cashew apple | 95.91% ± | 97.68% ± | 96.55% ± | 97.61% ± |
| extract of | 2.48 | 0.93 | 0.98 | 2.56 |
| Example 1 | | | | |
| | Concentration | | | |
| | 0.005% | 0.01% | 0.015% | 0.02% |
| | (0.05 mg/ml) | (0.1 mg/ml) | (0.15 mg/ml) | (0.2 mg/ml) |
| Reference | 107.71% ± | 88.34% ± | 46.60% ± | 27.16% ± |
| standard | 9.65 | 5.47 | 2.83 | 1.29 |

Cashew extract induced a cell viability of 95.91%±2.48 at the lowest concentration of 0.25% (2.5 mg/ml) and 97.61%±2.56 at the highest concentration of 1% (10 mg/ml), while that for the reference standard was 07.71%±9.65 at 0.005%, which reduced in a dose dependent manner to 27.16%±1.29 at 0.02%. There was no cytotoxicity observed. It was seen that viability of cells decreased with increase in concentration of the cashew apple extract.

### Example 4: Agar Diffusion Assay

An agar diffusion assay was carried out to study the reactivity by determining the inhibition zone due to the cashew apple extract of Example 1, on Murine Fibroblast cell line (3T3), in full compliance with the USP Guidelines for biological reactivity tests (U.S. Pharmacopoeia, Biological tests/(87) Biological tests, in vitro, Agar Diffusion Test. Pg 2525-2526). A total of three independent experiments were carried out. The Reactivity of the test sample was assessed by the measurement of the inhibition zone. The assay was carried out to study the effect of the sample for 24 hrs, using the Murine Fibroblast Cell Line (3T3). The investigational sample was tested neat. The assay was performed in 60 mm tissue culture grade plastic petridishes. The duration of the assay was 4 days. The cells were seeded into the petridishes and after they were sub-confluent they were treated with the test material for 24 hours.

After an exposure of 24 hours, the assay was terminated by addition of MTT (3-[4,5-Dimethylthiazol-2-yl]-2,5-diphenyltetrazolium bromide; Thiazolyl blue; Sigma M 5655). MTT is a water-soluble tetrazolium salt, which is converted to an insoluble purple formazan. This conversion is caused by active mitochondrial dehydrogenases of living cells. Dead cells do not cause this change. Thus, if the investigational sample causes cell death, this will be indicated by a clear zone of inhibition. The colour was allowed to develop overnight and the zone of inhibition was measured. Cashew apple extract of Example 1 was found to have no zone of inhibition at 100% concentration; there were no malformed or degenerated cells observed under specimen loading. Hence, it was classified as non-reactive. The positive control was found to have an average zone of inhibition of 1.05±0.05 cm. The negative control was found to have no zone of inhibition.

In brief the study yielded the following zones of inhibition as shown in Table 4 below.

TABLE 4
Zone of Inhibition (cm)

| | | Positive | Negative |
|---|---|---|---|
| | Cashew Extract | Control | Control |
| | Cashew apple | Positive | Negative |
| | extract of Example 1 | control | control |
| Experiment 1 | 0.00 | 1.01 | 0.00 |
| Experiment 2 | 0.00 | 1.11 | 0.00 |
| Experiment 3 | 0.00 | 1.03 | 0.00 |
| Mean ± SD | 0.00 | 1.05 ± 0.05 | 0.00 |

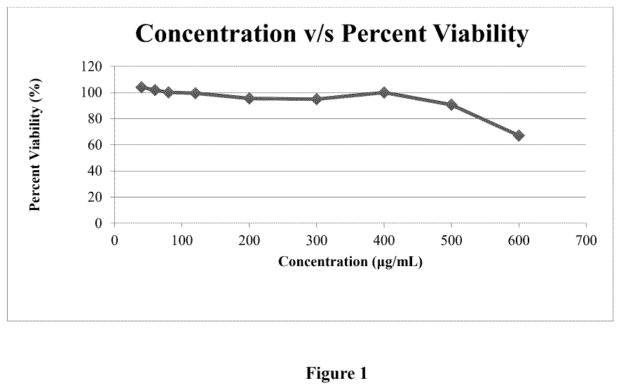

A graph was plotted for the absorbance (Y-axis) against the concentration of drug (X-axis) (FIG. 1). The IC50 was calculated as the drug concentration that is required to reduce the absorbance to half that of the control sample. The IC50 was found to be 600 μg/mL. The mean absorbance reading from the wells in columns 2 and 11 was used as a control. The cytotoxicity studies revealed that cashew apple extract is nontoxic to 3T3 fibroblast cells and is safe to be used in cosmetic products.

### Example 5: Differentiated Adipose Cell Efficacy Study

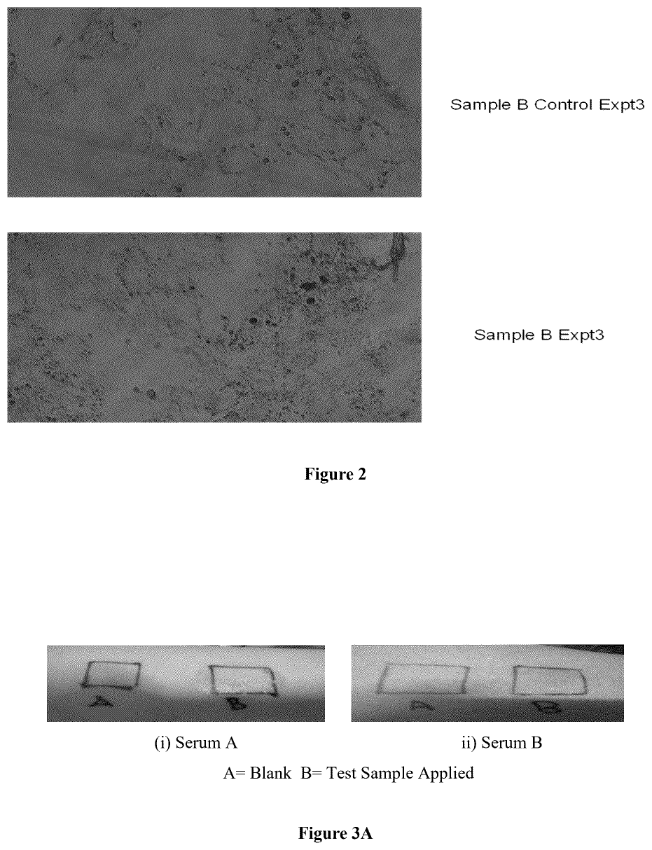

An in vitro assessment of the cashew apple extract of Example 1 was done on differentiated adipose cells for anti-cellulite activity. The objective of the anti-cellulite assay was to study the adipocyte lipolysis potential by determining the reduction in cell size due to the test samples, i.e. the cashew apple extract of Example 1 at 50% concentration. The assay was performed in tissue culture grade 96 well plates. The duration of the assay was 4 weeks. The undifferentiated stem cells were seeded into the 96 well plates and differentiated into adipocytes over a period of 3-4 weeks using differentiation protocol. After the cells had undergone differentiation, they were treated with the test samples for 24 hours. The assay was terminated by staining the cells with Oil Red O, solubilized and read at 450 nm on a plate reader. If the investigational sample has any lipolytic effect, the adipocytes will shrink leading to a reduction in the color. The percentage activity was calculated by percentage reduction in color, in comparison with the untreated control. The cashew apple extract of Example 1 was found to have an average percentage reduction in color of 5.57 at 50% concentration, indicative of reduced adipogenesis, i.e. reduction in cellulite. The results are shown in FIG. 2.

### Example 6: Sebutape Method

The Sebutape method was used to analyse the skin surface lipid before and after application of the compositions containing cashew apple extract. Sebutape® is an adhesive, white tape specifically designed for the collection of sebum. As sebum issues from the orifices, it is trapped in microcavities in the tape, yielding a pore pattern that can be quantified in regard to the number of spots, total area, and size distribution. It detects not only the amount of sebum but also the sebum pore structure of the skin. The area occupied by spots per cm² is a measure of sebum production, while the number of spots reflects actively secreting follicles. The Sebutape® is removed after one hour of application to the area of skin that needs to be analysed, and placed upon a black card for storage and analysis. Active pores (filled with sebum) block the light and form spots on the film, when viewed against a black background. In order to quantify skin pore widening, three-dimensional data of skin pore is produced by a stereoimage optical topometer (SOT). The sizes of follicular infundibulum are measured quantitatively, with reserved sebum by Sebutape®. This method is used for testing of cosmetic formulations, and the analysis of the tape using this method helps understand the impact of the formulation on the lipid structure of the skin.

Sebutape strip was applied on marked area of skin and kept intact with the skin for two time modules (after 20 minutes and 3 hours) and evaluated under microscope. Simultaneously, tape stripping method was used to evaluate the skin texture of the area. Quantitative image analysis of a photograph of the Sebutape strip placed against a black background allowed determination of the number and size of lipid spots.

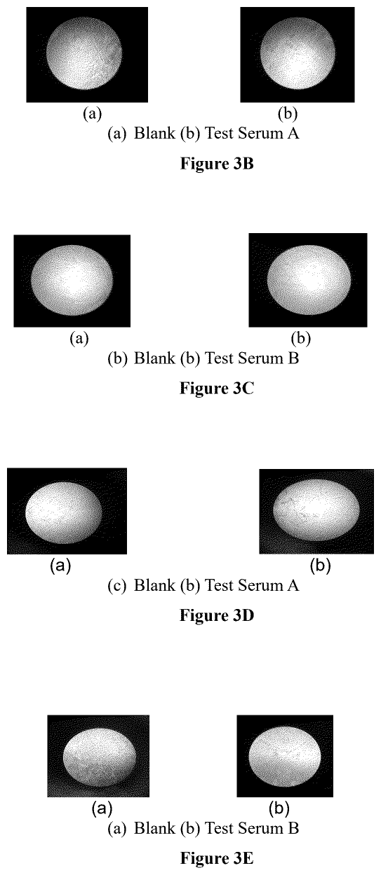

Serum A and Serum B (Formulation I) was tested on volunteers using Sebutape method, using a blank as control. It was observed that the skin pore size was reduced and the surface of the skin was found to be smoother and more subtle, and fibres were more meshed after application of the samples. The cashew apple extract (both Serum A and Serum B) was found to give a moisturizing and toning effect to the skin. It was also found to reduce the gap between the skin cells, and it helped remove only the fats. The study confirmed that the cashew apple extract of the present invention removes fat from skin without replacing the surface lipids. FIGS. 3A-3E provide testing and microscopic evaluation details for Serum A and Serum B (Formulation I) of Example 2.

### Example 7

The serums A and B of Example 2 were tested on human volunteers and it was observed that a 50% reduction in the thigh and arm area was attained after use of products for 15 days. The thigh and arms were measured before and after the application of the product twice a day. The observations for application to thighs are recorded in Table 5 below.

TABLE 5

| Days | Serum A | Serum B |
|---|---|---|
| Day 1 | 20.4 cm | 21.2 cm |
| Day 7 | 19.4 cm | 18.3 cm |
| Day 15 | 19.2 cm | 14.9 cm |

The Serum B was found to penetrate the skin better than Serum A, and worked on the targeted area without any irritation or discomfort throughout the application. The gap between the skin cells was found to reduce, and the skin appeared to be firmer and more moisturized, leading to a smooth and soft feel.

### Example 8: Preparation of Formulation II

Formulation II was prepared using components as shown in Table 6 below.

TABLE 6

| Phase | Ingredients | % w/w |
|---|---|---|
| A | Carbomer 940 | 0.3 |
| | Demineralised Water | 79.25 |
| | Sodium Gluconate | 0.2 |
| | Glycerine | 4 |
| B | Stearic Acid | 2.5 |
| | Cetostearyl Alcohol | 1.75 |
| | Isopropyl myristate | 4 |
| C | Triethanolamine | 0.3 |
| D | Caprylic/Capric Monoglyceride | 0.7 |
| | Piper nigrum Oil | |
| | Sophorolipid | |
| E | Cashew fruit extract of Example 1 | 5 |
| | Glycerine | |
| | Sodium Benzoate | |
| | Potassium Sorbate | |
| | Isopropyl Alcohol | |
| | Water | |

The formulation was prepared by first weighing and adding all Phase A ingredients into a mixing vessel. The carbomer was allowed to swell under low-speed stirring to avoid aeration. In parallel, Phase B ingredients were weighed and mixed separately. Both phases were then heated to approximately 70° C., after which Phase B was gradually added to Phase A with continuous stirring to ensure uniform mixing. Once the temperature reached 65° C., Phase C ingredient was added to the mixture and stirred thoroughly. The mixture was then cooled to approximately 40° C., at which point the Phase D and E ingredients were incorporated. The blend was stirred continuously until a homogeneous emulsion was obtained. The final product was a stable, cosmetically acceptable product suitable for topical application, and was packaged appropriately.

### Example 9: Anti-Tyrosinase Activity of Formulation II

Tyrosinase plays a vital role in the melanin pathway. The anti-tyrosinase activity of a test substance is determined by incubating the enzyme with the test and substrate. The uninhibited enzyme will oxidize the substrate tyrosine to DOPA-quinone, which can be detected spectrophotometrically at 450 nm. The resultant colour is inversely proportional to the anti-tyrosinase activity. Hence, higher the product formed, more intense is the color, and consequently, lesser is the inhibitory effect of test sample on the enzyme. The reference standard is run concurrently to comparatively assess the anti-tyrosinase activity, which is expressed as % activity per mg/ml.

The anti-tyrosinase activity of Formulation II of Example 8 was evaluated in vitro using a mushroom tyrosinase enzyme inhibition assay. The objective was to determine the potential of the Formulation II (test sample) to inhibit melanin synthesis, which is indicative of skin brightening efficacy.

The study was conducted using four concentrations of the test sample: 25 mg/ml, 50 mg/ml, 75 mg/ml, and 100 mg/ml. A reference standard (kojic acid) was tested at 0.01 mg/ml, 0.025 mg/ml, and 0.05 mg/ml for comparative purposes. Kojic acid concentrations typically used in cosmetics were used as the standard. Absorbance was measured at 450 nm, and percent tyrosinase inhibition was calculated. Brightening effect of the test versus sample was compared.

The cashew apple extract exhibited dose-dependent tyrosinase inhibition. The results are provided in Table 7 below.

TABLE 7

| SAMPLE | ANTI-TYROSINASE ACTIVITY (%) | | | |
|---|---|---|---|---|
| | Concentration of Formulation II | | | |
| | 2.5% | 5% | 7.5% | 10% |
| | (25 mg/ml) | (50 mg/ml) | (75 mg/ml) | (100 mg/ml) |
| Absorbance of | 24.24 ± 3.07 | 38.45 ± 7.02 | 43.74 ± 9.09 | 50.38 ± 2.81 |
| Formulation II | | | | |
| | Concentration of reference standard | | | |
| | 0.01 mg/ml | 0.025 mg/ml | 0.05 mg/ml | |
| Absorbance of | 49.80 ± 6.08 | 84.31 ± 5.06 | 95.30 ± 2.17 | |
| reference | | | | |
| standard | | | | |

The IC₅₀ value (concentration at which 50% inhibition is achieved) was calculated to be 98 mg/ml. These results demonstrate that Formulation II possesses measurable anti-tyrosinase activity, supporting its use in cosmetic formulations aimed at skin brightening, pigmentation control, and complexion enhancement.

### Example 10: Evaluation of Collagen Synthesis of Formulation II

Direct Red Assay was used to measure the collagen production by Formulation II of Example 8 (test sample). Sirius Red or Direct Red 80 is a specific dye which stains collagen and is a standard procedure in histology. It has been developed as a colorimetric dye-binding assay to directly quantify collagen. This was a modified protocol where the ability of the formulation to induce collagen synthesis in human Mesenchymal Stem Cells (hMSCs) was evaluated. After treatment with the test samples, the cells were stained with Direct Red, solubilized and read at 570 nm in a plate reader.

Human mesenchymal stem cells (hMSCs) were cultured under standard conditions and seeded into 24-well plates. Once the cells reached the appropriate confluence, they were treated with different concentrations of the test Formulation II, i.e.: 5 mg/ml, 10 mg/ml, 20 mg/ml and 40 mg/ml. A reference standard (ascorbic acid) was used at concentrations of 0.005 mg/ml, 0.01 mg/ml, 0.05 mg/ml, and 0.1 mg/ml, which is the concentration at which ascorbic acid is typically used in formulations. After 24 hours of incubation, the cells were washed with phosphate buffered saline (PBS) and fixed using Bouin's solution. The fixed cells were stained with Direct Red 80 dye for 1 hour. Excess dye was washed off with 0.1N HCl, and the bound dye was eluted using 0.1N NaOH:methanol solution. The absorbance of the solubilized dye was measured at 570 nm using a plate reader.

The results are shown in Table 8 below.

TABLE 8

| SAMPLE | COLLAGEN SYNTHESIS ( mg/ml) | | | |
|---|---|---|---|---|
| | Concentration of Formulation II | | | |
| | 0.5% | 1% | 2% | 4% |
| | (5 mg/ml) | (10 mg/ml) | (20 mg/ml) | (40 mg/ml) |
| Absorbance of | 0.158 ± | 0.165 ± | 0.16 ± | 0.143 ± |
| Formulation II | 0.033 | 0.038 | 0.034 | 0.018 |
| | Reference Standard concentration | | | |
| | 0.005 mg/ml | 0.01 mg/ml | 0.05 mg/ml | 0.1 mg/ml |
| Absorbance of | 0.119 ± | 0.110 ± | 0.095 ± | 0.086 ± |
| Reference | 0.0122 | 0.007 | 0.009 | 0.007 |
| Standard | | | | |

The results clearly demonstrate that Formulation II provides significantly higher collagen production in human mesenchymal stem cells, compared to the reference standard. The activity is most pronounced at 10 mg/ml, indicating an optimal concentration range for topical application, thereby supporting the anti-aging benefits of Formulation II via collagen synthesis.

### Example 11: Skin Irritation Test

Patch test is a technique used to determine the potential of a formulation to cause irritation of the skin. Irritants are substances that damage the skin by direct toxic action. The damage depends upon the nature of the irritant, its concentration and duration of exposure. Irritation is manifested as inflammatory responses such as erythema (redness), oedema (swelling), vesiculation, and finally an intense suppurative reaction without the involvement of immune system. To test the irritation potential of Formulation II of Example 8, patches containing the Formulation II were applied for a contact period of 24 hours, and skin reaction was assessed under a constant artificial daylight source. The marked sites were scored post 24 hours, after removal of the patches. Reactions such as erythema, dryness and wrinkling were scored on a 0 to 4-point scale, and oedema on another 0 to 4-point scale (Draize scale).

The test was conducted in accordance with IS 4011:2018 guidelines. The study was conducted on 26 healthy adult participants, both male and female, aged between 18 and 55 years (mean age 27.27 years). 0.04 ml of Formulation II was applied to filter paper discs placed in occlusive patch chambers. These were applied occlusively on the back of each participant. 1% sodium lauryl sulfate (SLS), an known irritant, was taken as the positive control, and 0.9% isotonic saline was used as the negative control. The patches were applied for 24 hours. At 48 hours post-application, clinical evaluations were conducted to assess skin reactions. Reactions were scored based on the Draize scale, where erythema/dryness/wrinkles were graded from 0 (no reaction) to 4 (severe reaction) and oedema was graded from 0 (no swelling) to 4 (severe swelling).

For Formulation II, 24 of 26 participants showed no reaction (score=0). Two participants showed very mild erythema (score=1), and none exhibited oedema. The results are provided in Table 9 below.

TABLE 9

| | Mean Erythema | Mean Oedema | Combined |
|---|---|---|---|
| Sample | Score | Score | Mean Score |
| Formulation II of | 0.08 | 0.00 | 0.08 |
| Example 8 | | | |
| Positive control | 2.12 | 0.96 | 3.08 |
| (1% SLS) | | | |
| Negative control | 0.00 | 0.00 | 0.00 |
| (0.9% isotonic | | | |
| saline) | | | |

As per IS 4011:2018, a combined mean score≤2.0 qualifies the substance as “Non-Irritant.” Therefore, Formulation II was conclusively classified as non-irritant.

### Example 12: Anti-Oxidant Assay

DPPH (2,2-diphenyl-1-picrylhydrazyl) assay was conducted to analyse the anti-oxidant activity of Formulation II of Example 8 (test sample). DPPH is a stable free radical with a deep violet color that exhibits strong absorbance at 517 nm. Upon interaction with an antioxidant, DPPH accepts an electron or hydrogen atom, becoming reduced and resulting in a measurable color change from violet to yellow. The extent of this discoloration is proportional to the antioxidant capacity of the test sample.

Formulation II of different concentrations i.e. 1% w/w, 5% w/w, and 10% w/w were mixed individually with DPPH and incubated for 30 minutes.

A reference standard (Kojic acid) was used at concentrations of 0.0001% w/w, 0.0005% w/w, 0.001% w/w, which are the standard concentrations at which it is used. These were mixed individually with DPPH and incubated for 30 minutes. Absorbance was measured at 517 nm using a UV-visible spectrophotometer. The results are provided in Table 10 below.

TABLE 10

| SAMPLE | Mean anti-oxidant activity | | |
|---|---|---|---|
| | Concentration of Formulation II (% w/w) | | |
| | 1 | 5 | 10 |
| Absorbance of | 46.38% ± 2.77 | 55.47% ± 2.90 | 66.79% ± 0.63 |
| Formulation II | | | |
| | Reference Standard concentration (% w/w) | | |
| | 0.0001 | 0.0005 | 0.001 |
| Absorbance of | 41.94% ± 5.83 | 87.46% ± 6.79 | 97.24% ± 6.46 |
| Reference | | | |
| Standard | | | |

The composition of the present invention demonstrated a mean antioxidant activity of 46.38%, indicating moderate free radical scavenging potential, in comparison to the reference standard.

### Example 13: Moisturisation Effect

To evaluate the moisturization effect of Formulation II of Example 8 (test sample), a scalar moisture checker was used. This is a non-invasive, electronic skin analysis device that quantitatively measures the moisture level on the skin surface.

The test sample was applied twice daily for a period of one month on the skin of healthy human volunteers. Measurements were taken daily for 15 days, with readings taken in the morning prior to application (baseline) and then at definite time intervals after application, using the scalar moisture checker. The results are provided in Table 11 below.

TABLE 11

| | | After | | | | |
|---|---|---|---|---|---|---|
| Day | Baseline | Application | 2 Hrs | 4 Hrs | 6 Hrs | 8 Hrs |
| 1 | 27.525 | 39.475 | 35.05 | 34.25 | 34.5 | 33.625 |
| 15 | 33.075 | 38.9 | 36.325 | 35.7 | 35.9 | 34.62 |

As can be seen above, the baseline moisture level of the skin improved from day 1 to day 15, due to once daily application of the Formulation II, as well as the moisture levels were seen to rise through the day, after application. These results confirm the ability of the composition to enhance and maintain skin hydration over time.

1. A composition for topical application comprising:
(i) cashew apple extract in an amount ranging from about 0.5% to about 50% by weight of the composition; and
(ii) one or more suitable excipients selected from the group consisting of solvents, polymers, oils, penetration enhancers, humectants, thickening agents, preservatives, antioxidants, chelating agents, fragrances, colours, emollients and pH adjusting agents,
wherein the composition is topically applied at least once daily for at least 15 days to the skin.

2. The composition according to claim 1, wherein the solvent is selected from the group comprising water, isopropyl alcohol, ethanol, benzyl alcohol, silicones, liquid paraffin, fatty acid esters, isopropyl palmitate, isopropyl myristate, propenediol, glycerine, caprylic/capric triglyceride, medium chain triglycerides, vegetable oil, diethyl succinate, diheptyl succinate, dihexyl succinate, diisooctyl succinate, neopentyl glycol diheptanoate, C₁₃-C₁₆ isoalkane, propylene glycol dibenzoate, isododecane, and a combination thereof.

3. The composition according to claim 1, wherein the solvent is present in an amount ranging from about 60% v/w to about 85% v/w of the total composition.

4. The composition according to claim 3, wherein the thickening agent is selected from the group comprising cetyl alcohol, stearyl alcohol, cetearyl alcohol, myristyl alcohol, beeswax, carnauba wax, candelilla wax, xanthan gum, guar gum, carrageenan, gum arabic, locust bean gum, pectin, agar, hydroxyethylcellulose, methylcellulose, carboxymethyl cellulose (CMC), hydroxypropyl methylcellulose (HPMC), carbomer, acrylic crosspolymers, sodium polyacrylate, corn starch, potato starch, aluminium starch, octenylsuccinate, sodium polyacrylate starch, silica, bentonite, magnesium aluminum silicate, gelatin, aloe vera and mixtures thereof.

5. The composition according to claim 1, wherein the thickening agent is present in an amount ranging from about ranging from 0.1% w/w to about 5% w/w of the total composition.

6. The composition according to claim 1, wherein the preservative is selected from the group comprising methyl paraben, ethyl paraben, propyl paraben, butyl paraben, benzoic acid, sodium benzoate, sorbic acid, potassium sorbate, diazolidinyl urea, imidazolidinyl urea, hydantoin, phenoxyethanol, benzyl alcohol, glyceryl ester, methylheptyl glycerine, caprylyl glycol, potassium hydrogen caprylhydroxamate, glyceryl undecylenate, glyceryl heptanoate, capryl hydroxamic acid, phenylethyl alcohol, ethylhexyl glycerine and mixtures thereof.

7. The composition according to claim 6, wherein the preservative is present in an amount of about 0.05% w/w to about 1% w/w of the total composition.

8. The composition according to claim 1, wherein the penetration enhancer is selected from the group comprising caprylic/capric monoglyceride, Piper nigrum (black pepper) oil extract, sophorolipid, isopropyl myristate, 2-pyrrolidone, propylene glycol, oleic acid, myristic acid, urea, isopropyl palmitate, ethanol, propenediol, sodium lauryl sulfate, benzalkonium chloride, maltodextrin, cyclodextrin, menthol, limonene, neem oil, eucalyptus oil, basil oil, nutmeg oil, sunflower oil, glycerine, urea, cetyl alcohol, cetostearyl alcohol, oleyl alcohol, betaine, mangiferrin and mixtures thereof.

9. The composition according to claim 8, wherein the penetration enhancer is present in an amount ranging from about 0.5% w/w to about 5% w/w of the total composition.

10. The composition according to claim 1, wherein the pH adjusting agent is selected from the group comprising citric acid, lactic acid, sodium hydroxide, potassium hydroxide, triethanolamine, sodium bicarbonate, arginine, aminomethyl propanol and mixtures thereof.

11. The composition according to claim 10, wherein the pH adjusting agent is triethanolamine.

12. The composition according to claim 1, wherein the chelating agent is present in an amount ranging from about 0.05% w/w to about 0.15% w/w of the total composition.

13. The composition according to claim 12, wherein the chelating agent is selected from the group comprising disodium EDTA, sodium gluconate and mixtures thereof.

14. The composition according to claim 1, wherein the emollient is selected from the group comprising shea butter, cocoa butter, lanolin, mineral oil, petrolatum, squalene, jojoba oil, castor oil, safflower oil, rosehip seed oil, avocado oil, caprylic triglyceride, capric triglyceride, isopropyl palmitate, isopropyl myristate, stearic acid, glyceryl stearate, octyl stearate, dimethicone, cyclomethicone, propylene glycol, stearic acid, palmitic acid, myristic acid, lauric acid and mixtures thereof.

15. The composition according to claim 14, wherein the emollient is present in an amount ranging from about 1% w/w to about 90% w/w of the total composition.

16. The composition according to claim 1, wherein the humectant is selected from the group comprising trehalose, glycerine, sorbitol, propylene glycol, hyaluronic acid, urea, honey, panthenol, lactic acid, glycolic acid, sodium gluconate, sodium lactate, butylene glycol, hexylene glycol and mixtures thereof.

17. The composition according to claim 16, wherein the humectant is present in an amount ranging from about 1% w/w to about 25% w/w of the total composition.

18. The composition according to claim 1 wherein the composition may be in the form of cream, gel, lotion, solution, tincture, ointment, mousse, serum, spray, mist oil, powder or paste.

19. The composition according to claim 18 wherein the composition is a serum.

20. The composition according to claim 1 wherein the composition is non-reactive to the skin and imparts anti-cellulite activity for skin sculpting.

21. The composition according to claim 1 wherein the composition provides anti-tyrosinase activity for skin brightening.

22. The composition according to claim 1 wherein the composition provides collagen stimulation to the skin for anti-aging effect.

23. The composition according to claim 1 wherein the composition exhibits free radical scavenging activity to provide antioxidant effect to the skin.

24. The composition according to claim 1 wherein the composition provides increased skin moisturization when applied at least once daily for at least 15 days.

25. A method for enhancing overall skin appearance comprising topically applying the composition of claim 1 to the skin at least once daily for a period of at least 15 days, wherein the method results in reduction in the appearance of cellulite, improvement in skin brightness, enhanced skin moisturization, stimulation of collagen production or reduced aging of the skin.
